(19) United States
(12) Patent Application Publication
(10) Pub. No.: US 2013/0184330 A1
(43) Pub. Date: Jul. 18, 2013

(54) COMPOSITIONS AND METHODS FOR INDUCING CANCER CELL DEATH

(71) Applicants: Theodore Lampidis, Miami, FL (US); Metin Kurtoglu, Miami Beach, FL (US)

(72) Inventors: Theodore Lampidis, Miami, FL (US); Metin Kurtoglu, Miami Beach, FL (US)

(73) Assignee: UNIVERSITY OF MIAMI, Miami, FL (US)

(21) Appl. No.: 13/817,207

(22) PCT Filed: Aug. 15, 2011
(86) PCT No.: PCT/US2011/047781

§ 371 Date: Mar. 28, 2013

Related U.S. Application Data
(60) Provisional application No. 61/374,711, filed on Aug. 18, 2010.

(51) Int. Cl. A61K 31/7004; A61K 31/198; A61K 31/713

(52) U.S. Cl. CPC A61K 31/7004; A61K 31/713; A61K 31/198
USPC 514/44 A; 514/23

(57) ABSTRACT

Described herein are pharmaceutical compositions including a sugar or mannose analog (e.g., 2-DG) and an inhibitor of at least one of PERK and GCN2 (e.g., an inhibitor of PERK and an inhibitor of GCN2) for treating cancer, and methods of treating cancer in a subject involving administration of these pharmaceutical compositions. Pharmaceutical compositions [or inducing death of cancer cells include a therapeutically effective amount of a pharmaceutical composition including: a pharmaceutically acceptable carrier, a sugar analog in an amount effective for inhibiting the growth of cancer cells, and an inhibitor of at least one of: PERK and GCN2 in an amount effective for blocking phosphorylation of eif2-1″t in the cancer cells, wherein the combined amounts of the sugar analog and the inhibitor of at least one of: PERK and GCN2 are sufficient for inducing death of the cancer cells.

### CROSS-REFERENCE TO RELATED APPLICATIONS

This application claims the benefit of Provisional Application Ser. No. 61/374,711 filed Aug. 18, 2010, which is herein incorporated by reference in its entirety.

### FIELD OF THE INVENTION

The invention relates generally to the fields of oncology, molecular biology, and medicine.

### BACKGROUND

With the recent surge in interest on tumor metabolism, attention has been given to inhibitors of glycolysis as a means of exploiting the Warburg phenomenon for therapeutic gain. 2-Deoxy-D-glucose (2-DG) has been extensively used to probe glucose uptake in a variety of cell types both in vitro and in vivo (Burckhardt and Stalder, Digestion, 1975, 12:1-8; Kurtoglu et al, Antioxid Redox Signal 2007, 9:1383-190; Duke et al., Lancet 1965, 2:871-876; Maher et al., Pancreas 2005, 30:e34-39). More recently, it has been reported that due to its inhibitory activity on glycolysis, 2-DG can be used to kill the slow-growing hypoxic population found in most solid tumors that are resistant to traditional chemotherapy which targets aerobic rapidly-proliferating cells (Boutrid et al., Invest Ophthalmol Vis Sci 2008, 49:2799-2805; Maher et al., Cancer Chemother Pharmcol 2004, 53:116-122). In vivo proof of principle that 2-DG targets the hypoxic cell population of solid tumors has also been recently reported in a transgenic model of retinoblastoma (Boutrid et al., Invest Ophthalmol Vis Sci 2008, 49:2799-2805; Houston et al., Clin Ophthalmol 5:129-137; Pina et al., Invest Ophthalmol Vis Sci 51:6149-6156). Moreover, a phase I trial using this strategy has been completed using 2-DG in combination with taxotere in patients with various tumor types (Raez et al., Journal of Clinical Oncology 2005, 23:1). Results indicate that 2-DGC is well-tolerated but due to the nature of the trial little can be said about efficacy.

In contrast to cells treated with 2-DG under hypoxic conditions, most tumor types under normoxia are growth inhibited but do not undergo cell death when treated with moderate doses of 2-DG (Kurtoglu et al., Antioxid Redox Signal 2007, 9:1383-1390). 2-DG is known to block, or interfere with different metabolic pathways which results in a cell switching from an anabolic to a catabolic phenotype to conserve energy and survive. It appears that most of the cell's response to interference with normal glucose metabolism converges on inhibition of protein synthesis. To achieve this, several possibilities exist each of which may be contributing to the growth inhibitory effects of 2-DG as follows: First, is the well known AMP-kinase pathway which can be activated as a consequence of 2-DG's activity on glycolysis if intracellular ATP levels are reduced enough, which appears to vary from cell type to cell type as well as depend on environmental growth conditions i.e. aerobic vs anaerobic. Activated AMPK leads to inhibition of protein synthesis through reduction in mTOR activity thereby slowing cell growth. Second, is a consequence of 2-DG interfering with oligosaccharide synthesis which results in endoplasmic reticulum (ER) stress-induced activation of the unfolded protein response (UPR) (Schroder et al., Mutat Res 2005, 569:29-63; Parodi et al., Annu Rev Biochem 2000, 69:69-93). This in turn leads to activation of the UPR signal transducer PERK which phosphorylates the mRNA translation initiation factor eif2-α resulting in lowering of cyclin D1 levels, cessation of the cell cycle and growth inhibition. Phosphorylation of eIF2-α leads to attenuation of mRNA translation, which is considered to be a cell survival mechanism by limiting the influx of new proteins into the ER and thereby reducing the buildup of misfolded proteins. EIF2-α phosphorylation can also occur through a PERK-independent mechanism via GCN2, another eIF2-α kinase, resulting in effective shut down of mRNA translation. GCN2 is known to be activated in response to glutamine depletion. This raises the possibility that lower pyruvate as a consequence of 2-DG's activity as a glycolytic inhibitor may induce mitochondria to use glutarnine as an energy source by a mechanism known as glutamrinolysis, thereby depleting cytoplasmic glutamine levels and activating GCN2. It thus remains unclear which pathway 2-DG induces to effectively inhibit tumor cell growth under normal oxygen conditions.

### SUMMARY

Described herein are pharmaceutical compositions including a sugar (e.g., mannose) analog (e.g., 2-DG) and an inhibitor of at least one of PERK and GCN2 (e.g., an inhibitor of PERK and an inhibitor of GCN2) for treating cancer, and methods of treating cancer in a subject involving administration of these pharmaceutical compositions. In the presence of oxygen, 2-DG inhibits growth of tumor cells, and as described herein, experiments were performed to understand whether this is due to 2-DG's effects on glycolysis and lowering ATP levels or interference with glycosylation leading to ER stress and an unfolded protein response. Overwhelming data evidences that growth inhibition is mainly due to 2-DG's effects on glycosylation leading to ER stress and inactivation of Eif2-α, a kinase in the unfolded protein response responsible for protein translation. The finding that growth inhibition is mainly due to 2-DG's effects on glycosylation leading to ER stress and inactivation of Eif2-α, and the realization that shutting down protein translation via this pathway essentially protects the cell from further ER stress, lead to the hypothesis that the growth inhibition response to ER stress is a protective mechanism for the cancer cell. Thus, if there was an interference with this protective mechanism, it was hypothesized that 2-DG-induced growth inhibition could be converted to 2-DG-induced cell death.

In the experiments described below, 2-DG resistance was shown to inversely correlate with resistance to glucose deprivation, suggesting that glucose deficiency and 2-DG are not the same entities. This suggestion is further supported by the differential effect of GCN2 on glucose deprivation vs. 2-DG treatment. While in the former condition, GCN2 is a proapoptotic molecule, the experiments described herein show that the same kinase is cytoprotective against 2-DG treatment. Not wishing to be bound to any particular theory, it is more than likely that the lack of oxygen in the second carbon of 2-DG underlies the contrast between its effects and that of glucose deprivation. This subtle variation in its structure renders 2-DG also as a mannose analog which should precede its activity as a glucose analog given that the former sugar is 50 times less than the latter one both in human plasma and culture conditions.

Accordingly, described herein is a pharmaceutical composition including a pharmaceutically acceptable carrier, a sugar analog (e.g., 2-DG, an analog of 2-DG, 2-fluoro-D-mannose (2-FM), an analog of 2-FM, 2-bromo-D-manose (2-BM), an analog of 2-BM, 2-chloro-D- mannose (2-CM), an analog of 2-CM, 2-deoxy-D-mannose (2-DM), an analog of 2-DM, 2-fluoro-glucose (2-FG), and an analog of 2-FG) in an amount effective for inhibiting the growth of cancer cells, and an inhibitor of at least one of: PERK and GCN2 in an amount effective for blocking phosphorylation of eif2-α in the cancer cells. In the pharmaceutical composition, the combined amounts of the sugar analog and the inhibitor of at least one of PERK and GCN2 are sufficient for inducing death of the cancer cells. The amount of the sugar analog effective for inhibiting the growth of cancer cells is sufficient for inducing ER stress in the cancer cells (e.g., as measured by ER stress assays). In one embodiment, the inhibitor of at least one of: PERK and GCN2 is a PERK inhibitor (e.g., an siRNA directed against PERK) and the sugar analog is 2-DG. In another embodiment, the inhibitor of at least one of PERK and GCN2 is a GCN2 inhibitor (e.g., an siRNA directed against GCN2; glutamnine; and an amino acid other than glutamine that inactivates or dephosphorylates GCN2) and the sugar analog is 2-DG. In a typical embodiment, the cancer cells are growing under normoxia. The composition can include an inhibitor of PERK (e.g., an siRNA directed against PERK) and an inhibitor of GCN2 (e.g., glutamine).

Also described herein is a method of treating cancer in a subject (e.g., a mammal) including: administering to the subject a therapeutically effective amount of a composition including a pharmaceutically acceptable carrier, a sugar analog (e.g., 2-DG, an analog of 2-DG, 2-FM, an analog of 2-FM, 2-BM, an analog of 2-BM, 2-CM, an analog of 2-CM, 2-DM, an analog of 2-DM, 2-FG, and an analog of 2-FG) in an amount effective for inhibiting the growth of cancer cells, and an inhibitor of at least one of PERK and GCN2 in an amount effective for blocking phosphorylation of eif2-α in the cancer cells, wherein the combined amounts of the sugar analog and the inhibitor of at least one of: PERK and GCN2 are sufficient for inducing death of the cancer cells. The amount of the sugar analog effective for inhibiting the growth of cancer cells is sufficient for inducing ER stress in the cancer cells (e.g., as measured by ER stress assays). In one embodiment, the inhibitor of at least one of: PERK and GCN2 is a PERK inhibitor (e.g., an siRNA directed against PERK) and the sugar analog is 2-DG. In another embodiment, the inhibitor of at least one of: PERK and GCN2 is a GCN2 inhibitor (e.g., an siRNA directed against GCN2, glutamine, and an amino acid other than glutamine that inactivates or dephosphorylates (GCN2) and the sugar analog is 2-DG. In a typical embodiment, the cancer cells are growing under normoxia. The composition can include both an inhibitor of PERK (e.g., an siRNA directed against PERK) and an inhibitor of GCN2 (e.g., glutamine).

Further described herein is a kit for treating cancer in a subject including: a composition including a pharmaceutically acceptable carrier, a sugar analog (e.g., 2-DG, an analog of 2-DG, 2-FM, an analog of 2-FM, 2-BM, an analog of 2-BM, 2-CM, an analog of 2-CM, 2-DM, an analog of 2-DM, 2-FG, and an analog of 2-FG) in an amount effective for inhibiting the growth of cancer cells, and an inhibitor of at least one of: PERK and GCN2 in an amount effective for blocking phosphorylation of eif2-α in the cancer cells, wherein the combined amounts of the sugar analog and the inhibitor of at least one of: PERK and GCN2 are sufficient for inducing death of the cancer cells; packaging; and instructions for use. The amount of the sugar analog effective for inhibiting the growth of cancer cells is sufficient for inducing ER stress in the cancer cells (e.g., as measured by ER stress assays). In one embodiment, the inhibitor of at least one of: PERK and GCN2 is a PERK inhibitor (e.g., an siRNA directed against PERK) and the sugar analog is 2-DG. In another embodiment, the inhibitor of at least one of: PERK and GCN2 is a GCN2 inhibitor (e.g., an siRNA directed against GCN2; glutamine; and an amino acid other than glutamine that inactivates or dephosphorylates GCN2) and the sugar analog is 2-DG. In a kit, the composition can include both an inhibitor of PERK (e.g., an siRNA directed against PERK) and an inhibitor of GCN2 (e.g., glutamine).

Unless otherwise defined, all technical terms used herein have the same meaning as commonly understood by one of ordinary skill in the art to which this invention belongs.

As used herein, “inhibitor of GCN2” and “GCN2 inhibitor” means any agent that inhibits GCN2 expression and/or activity.

By the terms “PERK inhibitor” and “inhibitor of PERK” is meant any agent that inhibits PERK expression and/or activity.

As used herein, the phrase “sensitizing the cancer cells to 2-DG” means increasing their sensitivity or toxic response to 2-DG under normoxia.

By the term “normoxia” is meant a state in which the partial pressure of oxygen is equal to that of air pressure at sea level, and the term “hypoxia” refers to decreased levels of oxygen below normal levels. The determination of normoxic and hypoxic conditions may be dependent on the characteristics and environment of the individual patient and/or of the individual tumor mass.

As used herein, “protein” and “polypeptide” are used synonymously to mean any peptide-linked chain of amino acids, regardless of length or post-translational modification, e.g., glycosylation or phosphorylation.

By the term “gene” is meant a nucleic acid molecule that codes for a particular protein, or in certain cases, a functional or structural RNA molecule.

As used herein, a “nucleic acid” or a “nucleic acid molecule” means a chain of two or more nucleotides such as RNA (ribonucleic acid) and DNA (deoxyribonucleic acid).

The terms “patient,” “subject” and “individual” are used interchangeably herein, and mean a mammalian (e.g., human) subject to be treated and/or to obtain a biological sample from.

As used herein, “bind,” “binds,” or “interacts with” means that one molecule recognizes and adheres to a particular second molecule in a sample or orgartism, but does not substantially recognize or adhere to other structurally unrelated molecules in the sample. Generally, a first molecule that “specifically binds” a second molecule has a binding affinity greater than about 10⁸ to 10¹² moles/liter for that second molecule and involves precise “hand-in-a-glove” docking interactions that can be covalent and noncovalent (hydrogen bonding, hydrophobic, ionic, and van der waals).

The term “labeled,” with regard to a nucleic acid, protein, probe or antibody, is intended to encompass direct labeling of the nucleic acid, protein, probe or antibody by coupling (i.e., physically or chemically linking) a detectable substance (detectable agent) to the nucleic acid, protein, probe or antibody.

When referring to a nucleic acid molecule or polypeptide, the term “native” refers to a naturally-occurring (e.g., a wild type, WT) nucleic acid or polypeptide.

As used herein, the terms “therapeutic,” and “therapeutic agent” are used interchangeably, and are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like.

The term “sample” is used herein in its broadest sense. A sample including polynucleotides, peptides, antibodies and the like may include a bodily fluid, a paraffin embedded specimen, a soluble fraction of a cell preparation or media in which cells were grown, genomic DNA, RNA or cDNA, a cell, a tissue, skin, hair and the like. Examples of samples include saliva, serum, tissue, skin, blood, and plasma. Samples can be fresh and/or frozen.

As used herein, the term “treatment” is defined as the application or administration of a therapeutic agent to a patient or subject, or application or administration of the therapeutic agent to an isolated tissue or cell line from a patient or subject, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Although compositions, kits, and methods similar or equivalent to those described herein can be used in the practice or testing of the present invention, suitable compositions, kits, and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a graph showing that 2-DG is more toxic in PERK−/− vs. PERK+/+MEFs,

FIG. 2 is a photograph of an immunoblot showing that in response to 2-DG phosphorylated Eif2-α is decreased in PERK −/− MEFs but still expressed.

FIG. 3 is a schematic illustration of the pathway in which GCN-2 phosphorylation is another way to phosphorylate and inhibit protein synthesis.

FIG. 4 is a graph and a photograph of an electrophoretic gel showing silencing of GCN2 increases 2-DG toxicity in Perk −/− MEFs.

FIG. 5 is a schematic illustration of the pathway by which excess glutamine can reverse 2-DG-induced GCN-2 phosphorylation thereby increasing cell death in PERK−/− MEF.

FIG. 6 is a graph showing that excess glutamine increases toxicity of 2-DG in Perk −/− but not PERK +/+.

FIG. 7 shows that 2-DG inhibits tumor cell growth via ER stress. (A) MDA-MB-435 cell line was treated with the indicated doses of 2-DG for 24 hours followed by protein extraction and western blot analysis of mTOR, AMPK and eif2-α phosphorylation as well as expression of GRP78 and CHOP. Actin was used as loading control. (B) MDA-MB-435 cells were treated with either 2-DG or 2-FDG for 48 hours at doses indicated. (C) MDA-MB-435 cells were treated with indicated doses of 2-DG in the presence of absence of 1 mM exogenous mannose for 48 hours followed by growth inhibition analysis. (D) GRP78 expression was analyzed by western blot in cells treated with increasing doses of either 2-DG or FDG. Actin was used as a loading control. (E) 24 hours following the same treatment conditions as in (D), protein was extracted for western blot analysis of GRP78 expression while actin was used as a loading control. Each panel is representative of at least 2 experiments * p<0.05, ** p<0.01.

FIG. 8 shows that knock-down or knock-out of PERK converts 2-DG-induced growth inhibition to cell death. (A) MDA-MB-435 cells were incubated with siRNA against either luciferase (as negative control) or PERK for 30 minutes and then incubated for 24 hours before addition of 2-DG. Following 24 hours of 2-D) treatment, protein was extracted for western blot analysis of PERK, phosphorylated eif2-α, GRP78, CHOP and cleaved caspase 3. Actin was used as a loading control. (B) MDA-MB-435 cells incubated with either siLuciferase or siPERK were then treated with 8 mM of 2-DG for 48 hours followed by cell death analysis. (C) Wild type (PERK+/+) and PERK knock-out (PERK−/−) MEFs were treated with indicated doses of 2-DG or 2-FDG for 48 hours followed by cell death analysis. (D) Wild-type and PERK knock-out cells were treated with 2-DG for 24 hours followed by western blot analysis of phosphorylated eif2-α. Actin was used as a loading control. Each panel is representative of at least 2 experiments. * p<0.05, ** p<0.01.

FIG. 9 shows that inhibition of GCN2 increases 2-DG-induced cell death in PERK-knock-out MEFs. (A) PERK knock-out MEFs (PERK−/−) were incubated with either siLuciferase or siGCN2 for 30 minutes and then incubated for 24 hours before addition of 2-DG. Following 24 hours of 2-DG treatment, protein was extracted for western blot analysis of phosphorylated and total (GCN2. Actin was used as a loading control. (B) PERK−/− cells incubated with either siLuciferase or siGCN2 were treated with 8 mM of 2-DG for 48 hours followed by cell death analysis. (C) PERK−/− cells growing in either normal growth medium (Glutamine 4 mM) or medium supplemented with exogenous glutamine (Glutanamine 8 mM) were treated with low or high doses of 2-DG for 24 hours followed by western blot analysis of phosphorylated GCN2, GRP78 or CHOP. Actin was used as a loading control. (D) PERK−/− cells growing in either 4 mM or 8 mM glutamine were treated with low or high-dose of 2-DG for 48 hours followed by cell death analysis. Each panel is representative of at least 2 experiments. * p<0.05, ** p<0.01.

FIG. 10 shows that 2-DG induces greater cell death in MEFs with non-phosphorylatable eif2-α (A) MEFs with wild type (S/S) or mutated (A/A) eif2-α were treated with the indicated doses of 2-DG for 24 hours followed by western blot analysis of GRP78, phospsphorylated eif2-α and cleaved caspase 3. Actin was used as a loading control. (B) Wild type (MEF eif2a S/S) and mutant (MEF eif2a A/A) cells were treated with increasing doses of 2-DG for 48 hours followed by cell death analysis.

FIG. 11 shows that 2-DG competes with both glucose and mannose to inhibit glycolysis and N-linked glycosylation respectively.

FIG. 12 is a graph showing that si RNA knockdown of PERK in pancreatic tumor cells converts 2-G growth inhibition to toxicity.

### DETAILED DESCRIPTION

Described herein are pharmaceutical compositions including a sugar or mannose analog (e.g., 2-DG) and an inhibitor of at least one of PERK and GCN2 (e.g., an inhibitor of PERK and an inhibitor of GCN2) for treating cancer, and methods of treating cancer in a subject involving administration of these pharmaceutical compositions. The realization that growth inhibition represents a mechanism by which cells survive treatment to 2-DG led to the investigation of which of the pathways described above play a prominent role in this biologic activity. By identifying this pathway, it was hypothesized that interference with its components may allow for converting 2-DG-induced growth inhibition to cell death in tumor cells growing under normoxia. In the experiments described herein, data is presented which indicate that ER stress leading to an UPR is the major mechanism responsible for 2-DG's inhibitory activity on tumor cell growth. It was also found that interference with the UPR and downstream pathways (eif2-α) can convert 2-DG's static effect to a toxic one in different cell types growing under normal oxygen conditions. The results described herein show promise for clinical application as agents that inhibit components of the UPR (e.g., PERK inhibitors, GCN2 inhibitors) were shown to induce killing of tumor cells.

The below described preferred embodiments illustrate adaptations of these compositions, kits and methods. Nonetheless, from the description of these embodiments, other aspects of the invention can be made and/or practiced based on the description provided below.

### Biological and Chemical Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001: and Current Protocols in Molecular Biology, ed. Ausubel et al., Greene Publishing and Wiley-Interscience, New York, 1992 (with periodic updates). Methods of preparing and sources for obtaining 2-DG and 2-DG analogs are known in the art, and are described, for example, in U.S. patent application Ser. No. 12/280,541 and U.S. Pat. No. 6,670,330.

### Pharmaceutical Compositions for Treating Cancer In a Subject

Pharmaceutical compositions described herein for inducing death of cancer cells include a therapeutically effective amount of a pharmaceutical composition including a pharmaceutically acceptable carrier, a sugar or mannose analog in an amount effective for inhibiting the growth of cancer cells, and an inhibitor of at least one of: PERK and GCN2 in an amount effective for blocking phosphorylation of eif2-α, such that the combined amounts of the sugar or mannose analog and the inhibitor of at least one of: PERK and GCN2, are sufficient for inducing death of cancer cells. In the compositions, the amount of the sugar or mannose analog effective for inhibiting cancer cell growth is sufficient for inducing ER stress in the cancer cells, typically as measured by one or more ER stress assays. Any suitable sugar or mannose analog can be used. Examples of sugar analogs include: 2-DG (and analogs thereof), 2-FM (and analogs thereof), 2-BM (and analogs thereof), 2-CM (and analogs thereof), 2-DM (and analogs thereof), and 2-FG (and analogs thereof).

In one embodiment, the inhibitor of at least one of: PERK and GCN2 is a PERK inhibitor and the sugar analog is 2-DG or a mannose analog effective in interfering with oligosachharide synthesis leading to ER stress. However, any suitable PERK inhibitor can be used. An inhibitor of PERK reduces the level of PERK in a cell and/or reduces the activity of PERK in a cell. Any agent that reduces the level of PERK in a cell and/or reduces the activity of PERK in a cell can be used. An inhibitor of PERK active to reduce the level of PERK protein in the cell may be an inhibitor of transcription and/or translation of PERK. In addition, an inhibitor of PERK active to reduce the level of PERK protein in the cell may stimulate degradation of the PERK protein and/or PERK encoding RNA. An inhibitor of PERK transcription and/or translation may be a nucleic acid-based inhibitor such as an antisense oligonucleotides complementary to a target PERK mRNA, as well as ribozymes and DNA enzymes which are catalytically active to cleave the target mRNA. One example of a PERK inhibitor is an siRNA directed against PERK. Small molecule inhibitors that inhibit PERK activity by altering its protein conformation or by interfering with essential protein-protein interactions may be used.

In another embodiment, the inhibitor of at least one of: PERK and GCN2 is a GCN2 inhibitor and the sugar analog is 2-DG or mannose analog effective in interfering with oligosachharide synthesis leading to ER stress. Any suitable GCN2 inhibitor can be used. For example, an siRNA directed against GCN2 can be used. Another example of a GCN2 inhibitor is glutamine. In some methods, a composition including both an inhibitor of PERK and an inhibitor of GCN2 is administered to a subject in need thereof. In such an embodiment, for example, the PERK inhibitor may be an siRNA directed against PERK, and the GCN2 inhibitor may be glutamine or an siRNA directed against GCN2.

### Methods of Treating Cancer In a Subject

Described herein are methods of treating cancer in a subject. Although the experiments described herein pertain to pancreatic cancer cells and melanoma cells, the methods described herein can be used to treat any cancer. A typical method includes administering to the subject a therapeutically effective amount of a composition including a pharmaceutically acceptable carrier, a sugar or mannose analog in an amount effective for inhibiting the growth of cancer cells, and an inhibitor of at least one of: PERK and GCN2 (e.g., a PERK inhibitor, a GCN2 inhibitor, a combination of PERK inhibitor and GCN2 inhibitor) in an amount effective for blocking phosphorylation of eif2-α, such that the combined amounts of the sugar or mannose analog and the inhibitor of at least one of: PERK and GCN2, are sufficient for inducing death of cancer cells. In a typical method, the amount of the sugar analog is effective for inhibiting cancer cell growth and is sufficient for inducing ER stress in the cancer cells as measured by ER stress assays. Any suitable sugar analog can be used that is able to produce ER stress at clinically achievable doses. Some examples of sugar analogs (but not limited to) include: 2-DG (and analogs thereof), 2-FM (and analogs thereof), 2-BM (and analogs thereof), 2-CM (and analogs thereof), 2-DM (and analogs thereof), and 2-FG (and analogs thereof). In the methods, any suitable GCN2 inhibitor and/or PERK inhibitors can be used. Based on the experimental data described herein, it has been found that GCN2 inhibitors work best when PERK is inhibited. In the methods described herein, the pharmaceutical compositions are able to induce death of cancer cells that are growing under normoxia, including cancer cells of a tumor in vivo.

The therapeutic methods of the invention in general include administration of a therapeutically effective amount of the compositions described herein to a subject (e.g., animal, human) in need thereof, including a mammal, particularly a human. Such treatment will be suitably administered to subjects, particularly humans, suffering from, or having, cancer or symptom thereof. The compositions herein may be also used in the treatment of any other disorders in which an excess of PERK signaling, expression, or activity may be implicated.

In one embodiment, the invention provides a method of monitoring treatment progress. The method includes the step of determining a level of diagnostic marker such as PERK (e.g., any target delineated herein modulated by a composition or agent described herein, a protein or indicator thereof, etc.) or diagnostic measurement (e.g., screen, assay) in a subject suffering from or susceptible to a disorder or symptoms thereof associated with cancer in which the subject has been administered a therapeutic amount of a composition as described herein for treating the disease or symptoms thereof. The level of marker determined in the method can be compared to known levels of marker in either healthy normal controls or in other afflicted patients to establish the subject's disease status. In preferred embodiments, a second level of marker (e.g., PERK) in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

Also described herein are diagnostic and theranostic methods useful to determine whether a tumor or other cancer includes cells susceptible to the treatment methods of the invention. The term “theranostics” generally refers to therapy-specific diagnostics, which is the use of diagnostic testing to diagnose the disease, choose the correct treatment regime for that disease, and monitor the patient response to therapy. Theranostic tests can be used to predict and assess drug response in individual patients, and are designed to improve drug efficacy by selecting patients for treatments that are particularly likely to benefit from the treatments. Theranostic tests are also designed to improve drug safety by identifying patients that may suffer adverse side effects from the treatment.

### Kits

Described herein are kits for treating cancer in a subject. A typical kit includes: a composition including a pharmaceutically acceptable carrier, a sugar or mannose analog (e.g., 2-DG) in an amount effective for inhibiting the growth of cancer cells (and/or inducing ER stress as measured by upregulation of GRP78, PERK phosphorylation, phosphorylation of eif2-α and/or protein synthesis inhibition or reduction), and an inhibitor of at least one of: PERK (e.g., siRNA directed against PERK) and GCN2 (e.g., glutamine) in an amount effective for blocking phosphorylation of eif2-α; packaging; and instructions for use. In the composition, the combined amounts of the sugar or mannose analog and the inhibitor of at least one of: PERK and GCN2, are sufficient for inducing death of cancer cells. In some embodiments, the composition includes an inhibitor of PERK and an inhibitor of GCN2. Optionally, kits may also contain one or more of the following: containers which include positive controls, containers which include negative controls, photographs or images of representative examples of positive results and photographs or images of representative examples of negative results.

### Administration of Pharmaceutical Compositions

The administration of a composition including a sugar analog (e.g., 2-DG) in an amount effective for inhibiting the growth of cancer cells (and/or inducing ER stress as measured by upregulation of GRP78, PERK phosphorylation, phosphorylation of eif2-a and/or protein synthesis inhibition or reduction), and an inhibitor of at least one of: PERK (e.g., siRNA directed against PERK) and GCN2 (e.g., glutamine) in an amount effective for blocking phosphorylation of eif2-α, for the treatment of cancer (e.g., pancreatic cancer) may be by any suitable means that results in a concentration of the therapeutic that is effective in ameliorating, reducing, or stabilizing a neoplasia. The sugar analog (e.g., 2-DG) and an inhibitor of at least one of: PERK (siRNA directed against PERK) and GCN2 (e.g., glutamine) may be contained in any appropriate amount in any suitable carrier substance, and are generally present in an amount of 1-95% by weight of the total weight of the composition. The composition may be provided in a dosage form that is suitable for local or systemic administration (e.g., intratumoral, parenteral, subcutaneously, intravenously, intramuscularly, or intraperitoneally). The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Compositions as described herein may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below), The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions described herein may be in a form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam- nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., 2-DG and a PERK inhibitor) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylceilulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, hurnmectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethyilcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active anti-neoplasia therapeutic substance). The coating may be applied on the solid dosage form in a similar manner as that described in Encyclopedia of Pharmaceutical Technology, supra. At least two anti-cancer therapeutics (e.g., a composition including 2-DG and a PERK inhibitor, as well as a second anti-cancer therapeutic) may be mixed together in the tablet, or may be partitioned. In one example, the first active anti-neoplasia therapeutic is contained on the inside of the tablet, and the second active anti-neoplasia therapeutic is on the outside, such that a substantial portion of the second active anti-neoplasia therapeutic is released prior to the release of the first active anti-neoplasia therapeutic. In one embodiment, an anti-cancer therapeutic can be administered with 2-DG and a PERK/GCN2 inhibitor.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment. Compositions as described herein can also be formulated for inhalation and topical applications. Optionally, an anti-cancer therapeutic may be administered in combination with any other standard anti-cancer therapy; such methods are known to the skilled artisan and described in Remington's Pharmaceutical Sciences by E. W. Martin. In one example, an effective amount of a sugar analog (e.g., 2-DG) and an inhibitor of at least one of: PERK (e.g., siRNA directed against PERK) and GCN2 (e.g., glutamine) is administered in combination with radiation therapy. Combinations are expected to be advantageously synergistic. Therapeutic combinations that induce apoptosis or other types of cell death of cancer cells are identified as useful in the compositions and methods described herein.

The therapeutic methods described herein in general include administration of a therapeutically effective amount of the compositions described herein to a subject (e.g., animal, human) in need thereof, including a mammal, particularly a human. Such treatment will be suitably administered to subjects, particularly humans, suffering from, having, susceptible to, or at risk for cancer. Determination of those subjects “at risk” can be made by any objective or subjective determination by a diagnostic test or opinion of a subject or health care provider. The methods and compositions herein may be also used in the treatment of any disorders in which PERK activity and/or expression may be implicated.

### Effective Doses

The pharmaceutical compositions described herein are preferably administered to a mammal (e.g., human) in an effective amount, that is, an amount capable of producing a desirable result in a treated mammal (e.g., inducing cancer cell death). Such a therapeutically effective amount can be determined according to standard methods. Toxicity and therapeutic efficacy of the compositions utilized in methods of the invention can be determined by standard pharmaceutical procedures. As is well known in the medical and veterinary arts, dosage for any one subject depends on many factors, including the subject's size, body surface area, age, the particular composition to be administered, time and route of administration, general health, and other drugs being administered concurrently.

### EXAMPLES

The present invention is further illustrated by the following specific examples. The examples are provided for illustration only and should not be construed as limiting the scope of the invention in any way.

### Example 1

### Converting 2-DG-Induced Growth Inhibition to Cell Death in Cancer Cells Growing Under Normoxia

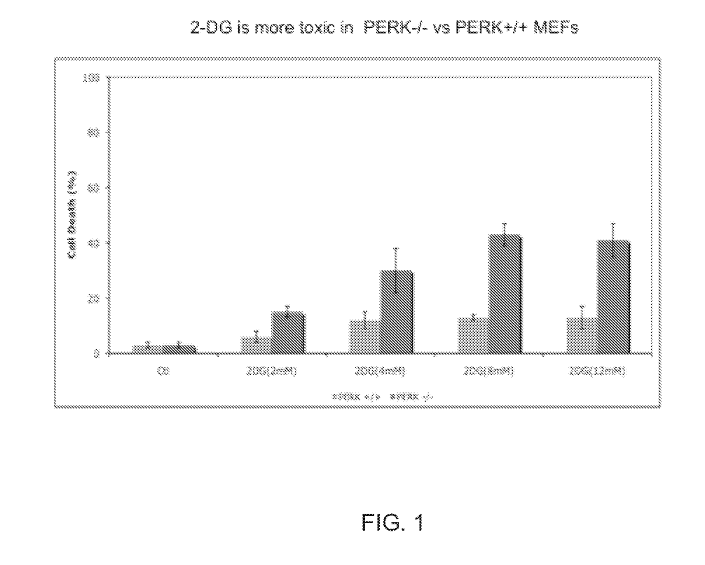

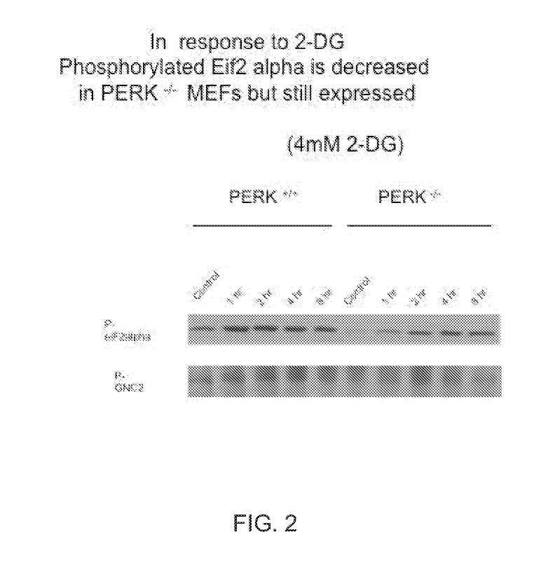

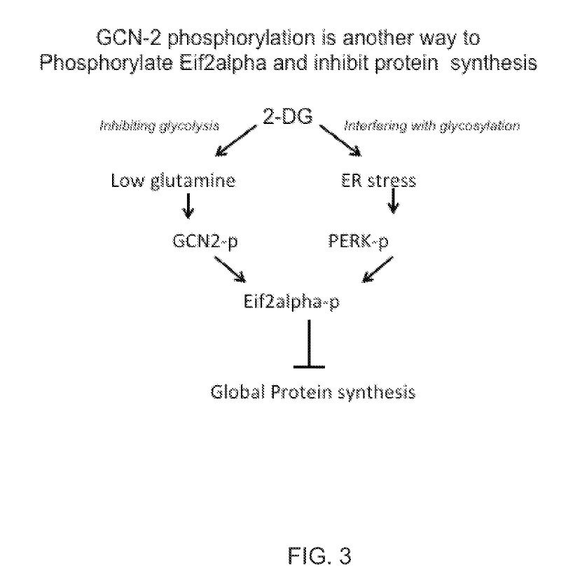

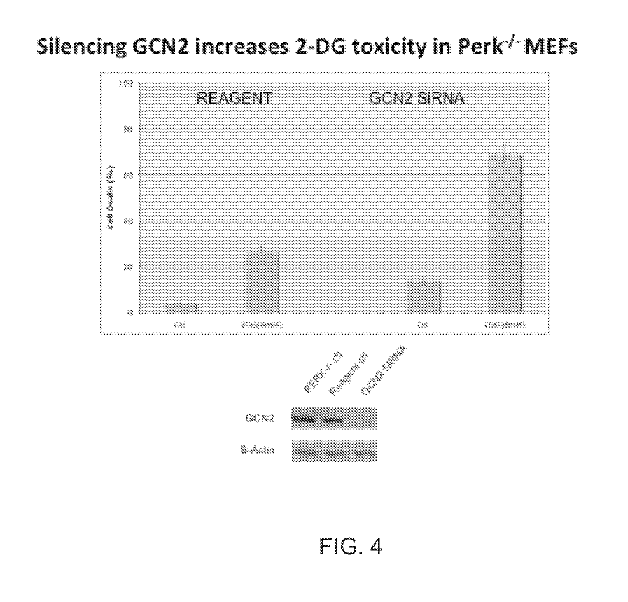

PERK, another kinase in the unfolded protein response pathway directly upstream of eif2-α, is one kinase that interacts with eif2-α and regulates its activity. It was reasoned that if PERK was blocked, this would allow eif2-α to continue to make proteins which would essentially increase ER stress leading to conversion of 2-DG-induced growth inhibition to cell death. This hypothesis was tested by using PERK^{-/-} mouse embryo fibroblasts and it was found that these cells were significantly more sensitive to 2-D)(than their PERK^{+/+} parental cell counterparts (FIG. 1). This mechanism was verified assaying the active and inactive state of eif2-α in the two cell pairs and found that indeed eif2-α was up-regulated in the PERK^{+/+} parental cell when treated with 2-DG but much less so in the PERK^{-/-} cell (FIG. 2). This confirmed that by knocking out PERK, the upstream regulator of eif2-α, it could not shut down its activity in response to ER stress as well as when it was intact. However, since some eif2-α activation was observed in the PERK^{-/-} cell line (albeit much less than in PERK^{+/+} cell line) (FIG. 2) it was hypothesized that perhaps GCN2, another kinase which is upstream of eif2-α and regulates its activity, could be responsible for the residual amount of eif2-α activity in PERK cell when challenged with 2-DG (FIG. 3). siRNA was used to block GCN2 and it was found that even greater 2-DG cell death in PERK^{-/-} cells when exposed to 2-DG as compared to when GCN2 was not blocked with SiRNA (FIG. 4).

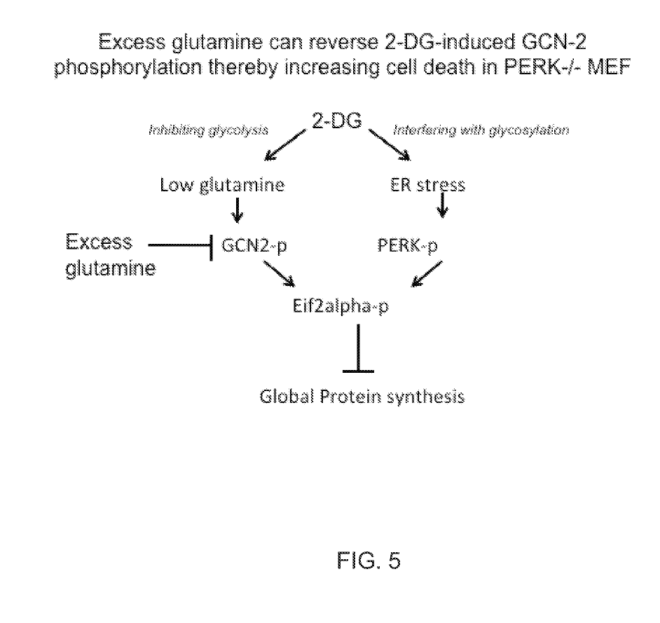

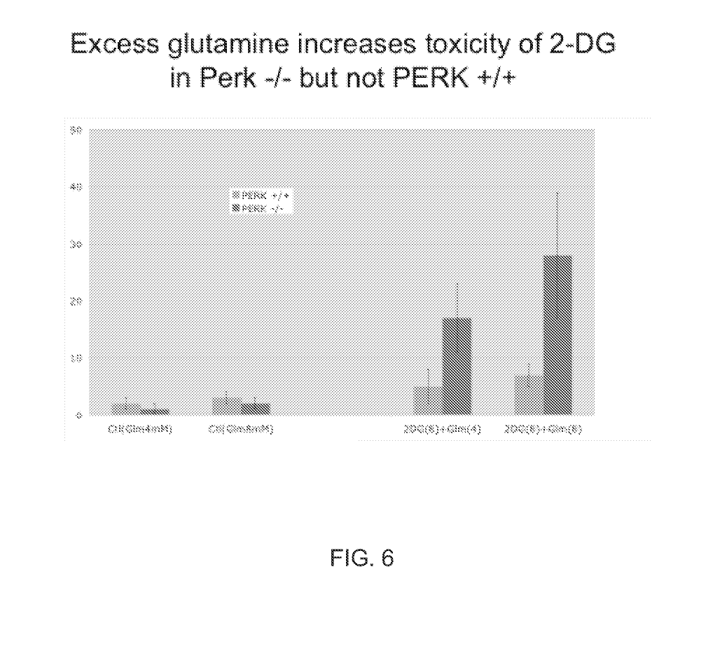

Knowing that GCN2 inactivates eif2-α activity in response to low amino acid content (FIG. 5), the hypothesis was tested whether increasing glutamine the medium could block its effect on Eif2-α and thereby increase 2-DG cytotoxicity. Indeed it was found that in the presence of 8 mM glutamine 2-DG toxicity in PERK^{-/-} cells is increased as compared to 2-DG treated cells in 4 mM glutamine (FIG. 6). Thus, these results were mimicked with siRNA blockage of GCN2 by merely supplying the cells with excess glutamine which makes this treatment possible for application to the clinic in converting 2-DG-induced growth inhibition to cell death.

In summary, the results show that in combination with eif2-α inhibition either in cells which have PERK knocked-out naturally and or by blocking GCN2 through presenting cells with excess glutamine ((8 mM) and greater), the static (growth inhibition) effect of 2-DG in cancer cells growing under normoxic conditions can be converted to a toxic one (cell death). Thus, PERK inhibitors and/or GCN2 inhibitors are useful in achieving the conversion of 2-DG induced growth inhibition to cell death in cancer cells growing under normoxia.

### Example 2

### PERK Inhibitors Convert 2-DG-induced Growth Inhibition to Tumor Cell Death

Inhibition of glucose metabolism has recently become an attractive target for cancer treatment. Accordingly, since 2-DG competes effectively with glucose, it has come under increasing scrutiny as a therapeutic agent. The initial response of a tumor cell to 2-DG treatment is growth inhibition which is thought to conserve energy and thereby protect the cell from its ATP-lowering effects as a glycolytic inhibitor. However, since 2-DG also mimics mannose causing it to interfere with N-linked glycosylation, the question is raised of how this sugar analog growth inhibits tumor cells and whether the mechanism can be manipulated to convert 2-DG-induced growth inhibition to cell death. Here, data is presented indicating that at 2-DG concentrations that can be achieved in human plasma without causing significant side-effects, it induces growth inhibition predominantly by interference with N-linked glycosylation, which leads to accumulation of unfolded proteins in the endoplasmic reticulum activating the UPR. Inhibition of PERK, a major component of the UPR, leads to conversion of 2-DG-induced growth inhibition to cell death. These results demonstrate that inhibition of PERK in combination with 2-DG is effective in killing tumor cells and therefore holds promise as a new therapeutic strategy.

### Methods and Materials

Cell types: Human tumor cell lines 1469 (pancreatic) and MDA-MB-435 (melanoma) were purchased from American Type Culture Collection (ATCC), and maintained in DMEM with 1 g/l of glucose (Mediatech). Mouse embryonic fibroblasts (MEF) that are PERK knock-out and its wild type pair were a kind gift from Dr. Mark Lehrman (University of Southwestern Texas, Dallas, Tex.). MEFs that have wild type eif2-α and eif2-α with its serine51 mutated to alanine were a kind gift from Dr. Glen Barber (University of Miami, Miami, Fla.). All MEFS were maintained in RPMI with 2 g/l of glucose (Invitrogen). All culture media were supplemented with 10% fetal bovine serum (FBS) (Invitrogen), penicillin/streptomycin (Invitrogen) and 0.1% Plasmocin (only for cell passage) (InvivoGen) unless otherwise specified. Cells were grown under 5% CO2 at 37° C.

Drugs and antibodies: 2-DG, 2-FDG, mannose and glutamine were purchased from Sigma-Aldrich. The following rabbit primary antibodies were from Cell Signaling: Grp78, PERK, GCN2, phospho-GCN2, phospho-eif2-α and cleaved caspase 3. Mouse anti-CHOP and anti-β-actin antibodies were from Cell Signaling and Sigma-Aldrich, respectively. Hlorseradish peroxidase (HRP)-conjugated anti-rabbit and anti-mouse IgG were purchased from Promega.

Immunoblotting analysis: Cells were seeded onto six-well plates and cultured for 18-22 h to reach 40-70% confluence. Following drug exposure for the indicated times, cells were harvested and lysed with the lysis buffer (100 mM Tris-HCl at pH 7.4, 1% SDS, phosphatase inhibitor cocktail 2 and protease inhibitor cocktail from Sigma-Aldrich). Protein concentrations of each sample were determined using a Micro BCA Protein Assay Kit (Thermo Scientific) according to the manufacturer's directions, and equal amounts of proteins were loaded onto 4-15% Tris-HCl gradient gels (except 12% gels for siRNA experiments) (Bio-Rad). After SDS-PAGE, proteins were transferred onto a polyvinylidene fluoride (PVDF) membrane (Millipore), blocked with 5% milk and probed with corresponding primary antibodies overnight (except 1 h for β-actin). The membrane was washed and probed with secondary antibodies for 1 h. Membrane was then incubated with SuperSignal West Pico or Femto Chemiluminescent Substrate (Thermo Scientific) and signals were visualized on Blue Lite Autorad Films (ISCBioExpress). All primary antibodies were used at 1:1000 dilution except for Atg7 (1:300) and β-actin (1:10000), and the secondary antibodies were used at 1:10000. Representative blots from at least three independent experiments were shown unless otherwise indicated.

Growth inhibition and Cytotoxicity assays: Cells were seeded onto 24-well plates and cultured for 18-22 h. After drug exposure for 48 hours, attached cells and their respective culture media were collected and centrifuged at 400 g for 5 min. The pellets were then resuspended in Hanks Balanced Salt Solution (HBSS) (Mediatech) and analyzed with Vi-Cell cell viability analyzer (Beckman Coulter) based on trypan blue exclusion. Results were shown as the percentages of dead cells out of total cells counted. Data were the averages of triplicate samples +SD from one representative experiment out of at least three independent analyses unless otherwise indicated.

siRNA transfection: Cells were seeded into 25-cm² flasks and cultured for 24 h to reach ˜60% confluence using antibiotics-free media. Then, cells were transfected with anti-Luc siRNA-1 (targeting luciiferase) or ON-TARGETplus SMARTpool siRNA against PERK or GCN2 using the DharmaFECT siRNA transfection reagent #2 (Dharmacon). Twenty-four hours after transfection, cells were collected and re-seeded onto 6-well or 24-well plates and drug-treated for immunoblotting or cytotoxicity analyses, respectively.

Statistical analysis: Data were compared using two-tailed paired Student's t-test, and P value less than 0.05 was considered significant.

### Results

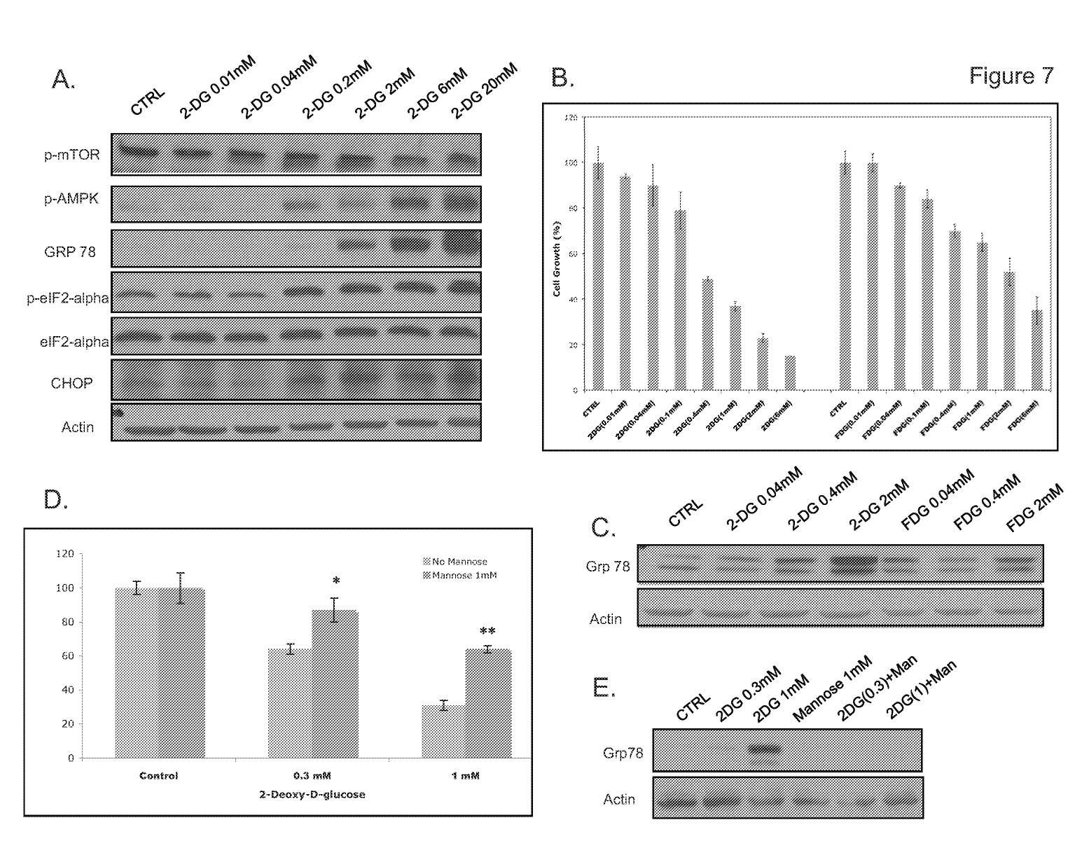

ER stress is a major mechanism by which 2-DG induces growth inhibition in tumor cells treated under normoxia. As indicated in FIG. 7A, 2-DG at low doses effects both ATP and ER sensing pathways. At 2-DG doses (0.04 to 0.3 mM) where growth inhibition is observed (FIG. 7B), AN MPK is detected to be phosphorylated (0.2 mM) (FIG. 7A). Similarly, a correlation is found between induction of ER, as detected by increased levels of the UPR folding protein chaperone GRP 78, and growth inhibition when MBA 435 human melanoma cells are treated with 2-DG under aerobic conditions. Moreover, the translation initiation factor, eIF2a, which responds to ER stress, is found to be phosphorylated (inhibited) at 2-DG doses where growth inhibition as well as increases in GRP 78 are significant (0.2 mM and 2 mM) (FIG. 7A and 7B). Thus, stimulation of either or both ATP and or ER sensing pathways may be contributing to 2-DG-induced growth inhibition. However, 2-fluoro-deoxy-D-glucose (2-FDG), another analog of glucose reported to be more potent in inhibiting glycolysis but less potent in interfering with glycosylation than 2-DG (2, 15, 16), showed significantly less growth inhibitory activity than 2-DG which correlated with less induction of the UPR marker GRP 78 at equivalent doses, i.e. 0.3 mM and 2 mM (FIGS. 7B and 7C). Moreover, addition of exogenous mannose which was previously reported to reverse the effects of 2-DG on ER stress but not ATP (10), reverses a significant amount of the growth inhibition and ER stress induced by 2-DG alone (FIGS. 7D and 7E). Taken together, these results indicate that 2-DG's induction of ER stress is a major mechanism by which cells are growth inhibited.

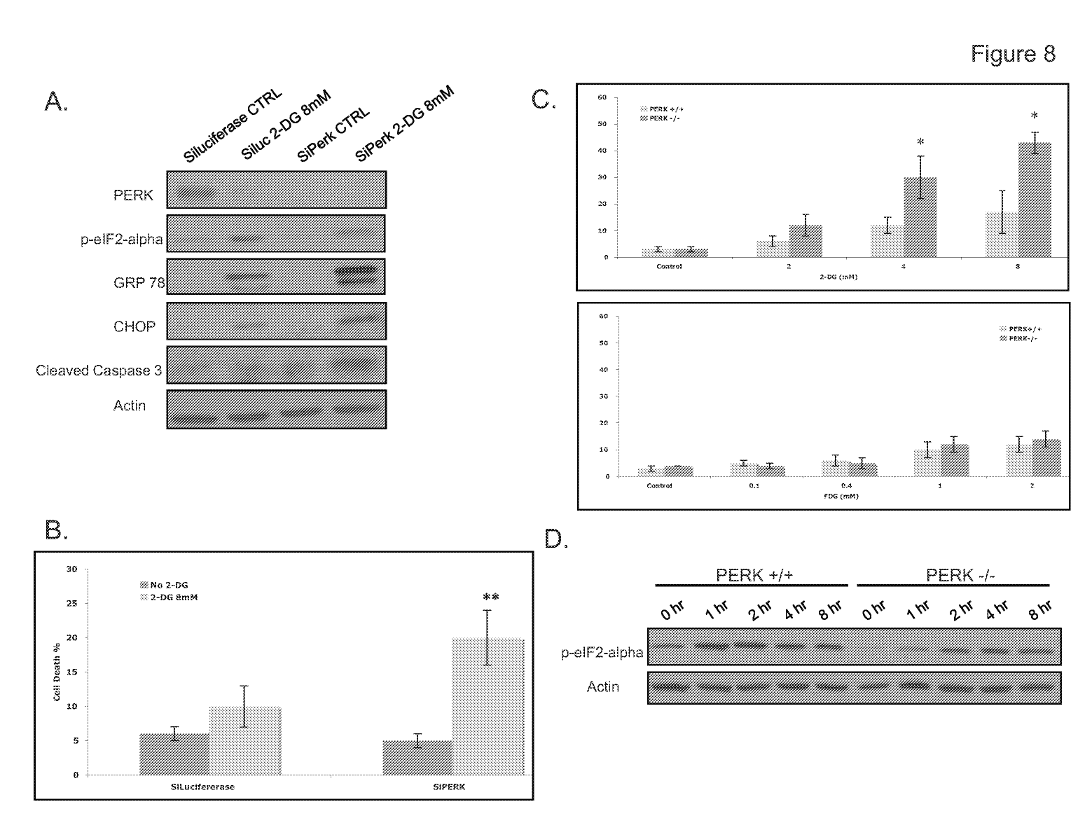

siRNA directed against PERK increases UPR markers and UPR-mediated cell death in human melanoma MDA-MB-435 cells treated with 2-DG. MBA-435 cells were transfected with siRNA directed against PERK. In FIGS. 8A and 8B, the results demonstrate that 2-DG induced CGRP78 and CHOP as well as toxicity is significantly increased in cells where PERK is knocked-down as compared to controls. These experimental results support the hypothesis that inactivating PERK leads to increased toxicity of 2-DG in tumor cells growing under oxygen.

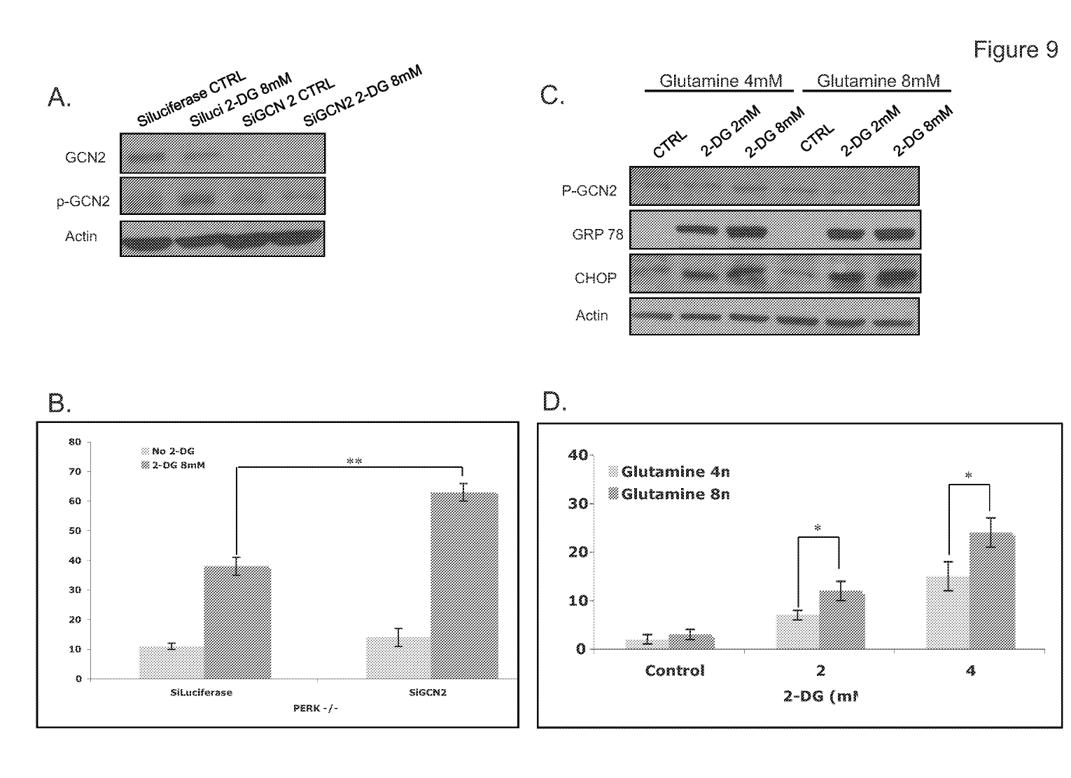

PERK−/− mouse embryo fibroblasts are more susceptible to the toxic effects of 2-DG than PERK+/+ cells which correlates with increased UPR-mediated cell death. Similar to tumor cells where PERK is inhibited by small molecules or siRNA, mouse embryo fibroblasts with deletions in PERK (PERK −/− cells) are found to be more sensitive to the toxic effects of 2-DG than their PERK +/+ counterparts (FIG. 8C). In contrast, 2-FDG, an analog of glucose that is less effective than 2-DG) (in causing ER stress and an UPR, yields very little or no toxicity in PERK −/− cells at doses that do not induce ER stress but block glycolysis effectively. Moreover, PERK−/− cells display significantly lower levels of phosphorylated eif2-α than their PERK +/+ counterparts when measured as a function of time of treatment with 2-DG (FIG. 8D). This result is in agreement with previous findings that PERK plays a major role in 2-DG mediated eif2-α phosphorylation. However, eif2-α phosphorylation was found to increase as a function of time in PERK−/− cells (albeit less than that in PERK+/+ cells) indicating that 2-DG induces eif2-α phosphorylation also through a mechanism independent of PERK. In this regard, it is known that eif2-α can respond to stress signals induced by lowered amino acid content namely thru phosphorylation of GCN2 another eif2-αphosphorylating kinase. In FIGS. 9A and 9B, data is presented which shows 2-DG cytotxicity is increased in PERK −/− cells when GCN2 is knocked-down with siRNA specific to this protein.

GCN2 is thought to respond to depletion of amino acids and thereby shuts down further protein synthesis by inhibiting eif2-α. It was therefore hypothesized that addition of exogenous glutamine to 2-DG treated PERK−/− cells would block activation of GCN2 which would in turn allow eif2-α to produce more proteins that would lead to increased ER stress and UPR-mediated cell death. This hypothesis is supported by the results in FIGS. 9C and 9D which show increased toxicity in 2-DG treated PERK −/− cells when co-treated with exogenous glutamine as compared to cells treated with normal glutamine levels. When grown in 4 mM glutamine, 2-DG at doses of 2 and 8 mM, induces phosphorylation of GCN2, however when exogenous glutamine is increased to 8 mM, 2-DG induced GCN2 phosphorylation is found to be attenuated (FIG. 9C). Furthermore, addition of exogenous glutamine reduces eif2-α phosphorylation correlating with increased ER stress and UPR-mediated cell death as assayed by GRP78 and CHOP levels (FIG. 9C). Thus, in addition to PERK, GCN2 appears to play a protective role against 2-DG induced cell death.

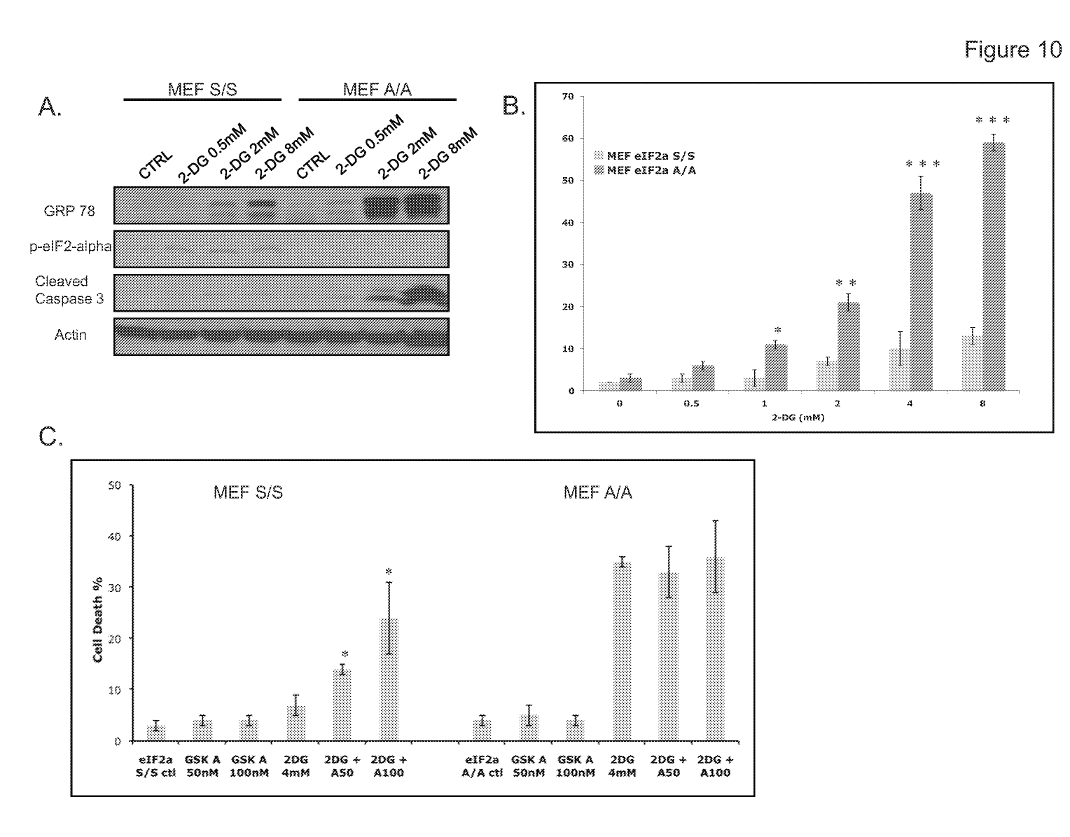

Genetic inhibition of eif2-α phosphorylation in MEFs renders cells hypersensitive to 2-DG. Since both PERK and GCN2 appear to protect the cell against 2-DG toxicity by inhibiting eif2-α, the effects of 2-DGC were examined in a mouse embryo fibroblast that contains a mutant form of eif2-α whose active site cannot be phosphorylated (inhibited). In FIGS. 10A and 10, it can be seen that this mutant cell line is more sensitive to the toxic effects of 2-DG than it parental counterpart expressing a normal eif2-α.

### Discussion

In the experiments described above, it was shown that mannose reverses a significant amount of 2-DC-induced growth inhibition which correlates with its reversal of the UPR marker, Grp78. A possible explanation for why 2-DG interferes with N-linked glycosylation more effectively when compared to its ability to inhibit glycolysis, may be the lower extracellular concentration of mannose as compared to glucose thereby stoichiometrically favoring 2-DG's interference with N-linked glycosylation as opposed to glycolysis. Interestingly, mannose reverses growth inhibition almost completely when cells are treated with lower doses of 2-ID) while at increased 2-DGC doses, reversal by mannose is incomplete (FIGS. 7D and 7E). This implies that at higher doses of 2-DG, pathways other than N-linked glycosylation, i.e. glycolysis and/or pentose-phosphate shunt may be contributing to the growth inhibitory effects of 2-DG. Additionally, support that PERK-inhibition leads to 2-DG induced cell death is demonstrated by increased cytotoxicity in cells transfected with siRNA-directed against PERK as well as in PERK−/− MEFs (FIG. 8).

In conclusion, it was found that 2-DG interferes with both glycolysis and N-linked glycosylation activating respective response pathways involved with protecting tumor cells from dying. However, the data strongly implicate interference with N-linked glycosylation rather than glycolysis as the predominant means by which 2-DG induces growth inhibition mediated through the UPR. Therefore, by blocking a critical component of the UPR, PERK, a major mechanism responsible for cancer cell protection from ER stress is overcome and 2-DG's effects become toxic. Overall, the data indicate that inactivating PERK combined with low (clinically achievable) doses of 2-DG offers a novel treatment strategy which may be universally applicable in a wide variety of tumor cell types growing under aerobic conditions.

### Example 3

### siRNA Knockdown of PERK In Pancreatic Tumor Cells Converts 2-G Growth Inhibition To Toxicity

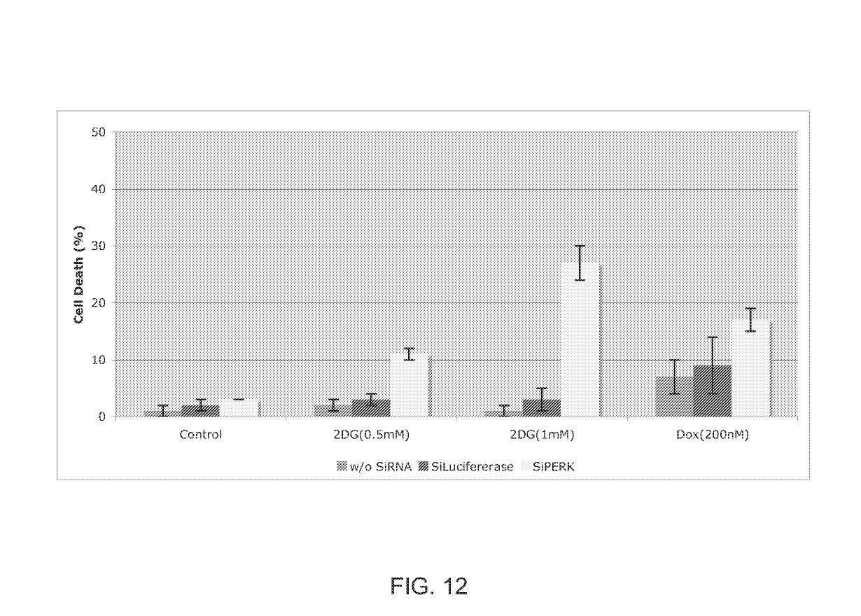

The hypothesis described herein was tested in cancer cells. SiRNA was used to knock-down PERK and the data (see FIG. 12) clearly show this leads to 2-DG induced toxicity in cancer cells.

### Example 4

### Agents That Inhibit PERK and/or GCN2 Result in Conversion of 2-DG Growth Inhibition To Cell Death For Clinical Applications

2-DG is unique in that it mimics both glucose and mannose. As an analog of glucose it inhibits glycolysis whereas as an analog of mannose it interferes with N-linked glycosylation. Both actions could account for its growth inhibitory properties. A major aim of the experiments described herein was to determine the pathway by which this occurs and by manipulating it, either genetically or chemically, convert 2-DG induced growth inhibition to cell death in numerous tumor types growing under normoxia. The results described herein favor interference with N-linked glycosylation leading to ER stress as the predominant mechanism. Once ER stress reaches a critical level, PERK, a transducer of the UPR acts as a protective mechanism by shutting down eif2-u, an initiator of protein translation, effectively reducing the number of proteins entering the ER. Alternatively, eif2-c can be phosphorylated by GCN2 which can also contribute to lowering ER stress and increasing survival in 2-DG-treated cells. Therefore, a goal is to form a preclinical basis for identifying metabolic targets such as PERK and/or GCN2, as well as others, and compounds that inhibit them, which result in conversion of 2-DG growth inhibition to cell death for clinical application.

The results from these experiments enable the use of 2-DG from solely killing hypoxic portions of tumors to targeting the entire tumor regardless of its environmental conditions, i.e. oxygen levels. Because increased tumor glucose metabolism is an inherent property of most if not all major oncogenes, targeting tumor glucose metabolism with 2-DG offers the possibility of providing a universal approach to treating cancer regardless of its oncogenic profile.

In addition to the findings that 2-DG induced ER stress appears to be the major pathway by which it growth inhibits tumor cells, another innovative component of the compositions and methods described herein is the use of agents that inhibit PERK or GCN2 to extend the toxicity of 2-DG to the normoxic portions of solid tumors. Moreover, based on the experience with 2-DG and 2-FDG as well as other specific inhibitors of either glycosylation or glycolysis, other tumor metabolic pathways may be identified that when targeted, will convert 2-DG induced growth inhibition to cell death.

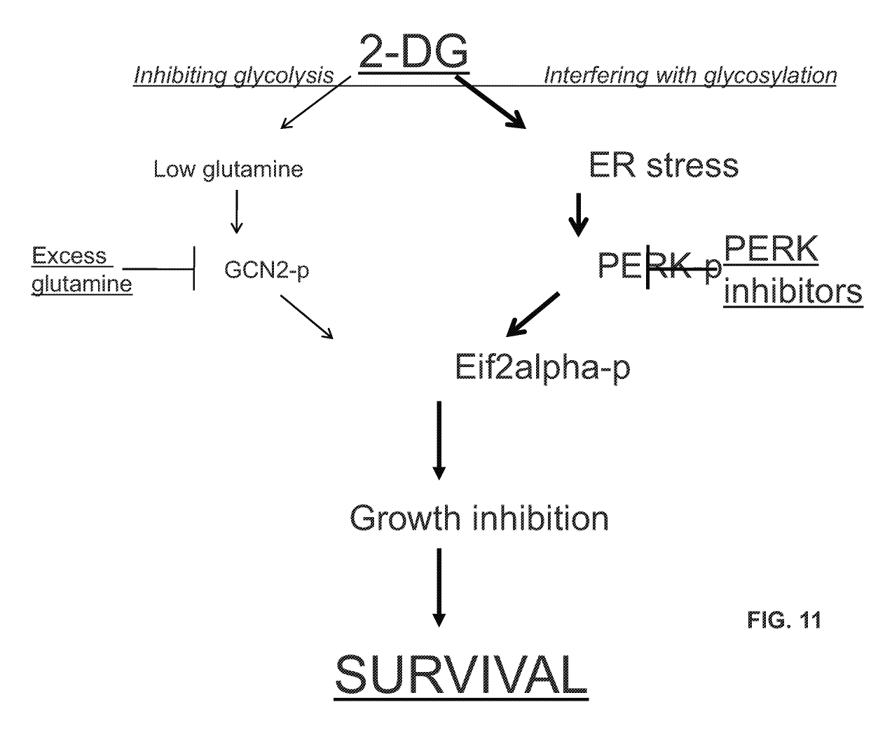

Referring to FIG. 11, since extracellular as well as intracellular concentrations of mannose are at least 10-fold less than glucose, 2-DG at clinically achievable doses interferes with N-linked glycosylation more readily than glycolysis. Therefore, 2-DCG-induced growth inhibition in tumor cells is predominantly a consequence of interference with N-linked glycoslyation that results in ER stress. When ER function is perturbed, eif2-α becomes phosphorylated via PERK to prevent further protein loading into ER, which suppresses proliferation and allows the cell to recover from ER stress. Therefore, inhibition of PERK increases 2-DG-induced ER stress and consequently converts growth inhibition to cell death. When the PERK pathway is inhibited in tumor cells treated with 2-DG, eif2-α can still be phosphorylated by GCN2. It was hypothesized that by inhibiting glycolysis, 2-DG lowers intracellular glutamine and thereby activates GCN2. Overall, inhibition of PERK and to a lesser extent GCN2 converts 2-DG-induced growth inhibition to cell death in tumor cells growing under normal oxygen levels. In FIG. 11, the consequence of 2-DG's interference with n-linked glycosylation is shown to lead to ER stress which in turn provokes a UPR response. Once ER stress reaches a critical level, PERK, a transducer of the UPR acts as a protective mechanism by phosphorylating and therby shutting down Eif2-α, the initiator of protein translation. This activity of PERK in stopping protein synthesis is thought to spare the ER of any further load of new proteins for folding thereby reducing ER stress and providing time for the cell to recover. PERK phosphorylation of eif2-α which in turn lowers the levels of cyclin D1, as well as other proteins necessary for cell growth, has been identified as the mechanism by which ER stress leads to growth inhibition.

Further support that PERK-inhibition leads to 2-DG induced cell death was demonstrated by increased cytotoxicity in cells transfected with siRNA-directed against PERK as well as in PERK−/− MEFs. Although eif2-α phosphorylation is found to be lower in PERK−/− as compared to PERK+/+ cells when treated with 2-DG, nevertheless the former cells had a temporal increase in phosphorylated eif2-α levels. This observation indicates that pathways other than PERK play a role in phosphorylating eif2-α by 2-DG which may also influence cell survival. In this regard, it is known that eif2-α can also be phosphorylated by GCN2, which is activated following intracellular glutamine depletion. Thus, it was found that 2-DG induced cell death is increased in PERK −/− cells by transfecting with siRNA directed against GCN2 or by increasing the amount of glutamine (FIG. 9). The observation that under similar conditions, 2-DG toxicity is not increased in PERK+/+ cells, indicates that GCN2 may play a secondary role to PERK in protecting cells against 2-DG induced-cell death.

In conclusion, it was found that 2-DG interferes with both glycolysis and N-linked glycosylation activating respective response pathways involved with protecting tumor cells from dying. However, the data described herein strongly implicate interference with N-linked glycosylation rather than glycolysis as the predominant means by which 2-DGC induces growth inhibition mediated through the UPR. Therefore, by blocking a critical component of the UPR, PERK, a major mechanism responsible for cancer cell protection from ER stress is overcome and 2-DG's effects become toxic.

### Other Embodiments

Any improvement may be made in part or all of the compositions, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A pharmaceutical composition comprising a pharmaceutically acceptable carrier, a sugar analog in an amount effective for inhibiting the growth of cancer cells, and an inhibitor of at least one of: PERK and GCN2 in an amount effective for blocking phosphorylation of eif2-α in the cancer cells, wherein the combined amounts of the sugar analog and the inhibitor of at least one of: PERK and GCN2 are sufficient for inducing death of the cancer cells.

2. The pharmaceutical composition of claim 1, wherein the amount of the sugar analog effective for inhibiting the growth of cancer cells is sufficient for inducing endoplasmic reticulum (ER) stress in the cancer cells as measured by ER stress assays.

3. The pharmaceutical composition of claim 1, wherein the sugar analog is selected from the group consisting of: 2-Deoxyglucose (2-DG), an analog of 2-DG, 2-fluoro-D-mannose (2-FM), an analog of 2-FM, 2-bromo-D-manose (2-BM), an analog of 2-BM, 2-chloro-D- mannose (2-CM), an analog of 2-CM, 2-deoxy-D-mannose (2-DM), and analog of 2-DM, 2-fluoro-glucose (2-FG), and an analog of 2-FG.

4. The pharmaceutical composition of claim 1, wherein the inhibitor of at least one of: PERK and GCN2 is a PERK inhibitor and the sugar analog is 2-DG.

5. The pharmaceutical composition of claim 4, wherein the PERK inhibitor is an siRNA directed against PERK.

6. The pharmaceutical composition of claim 1, wherein the inhibitor of at least one of: PERK and GCN2 is a GCN2 inhibitor and the sugar analog is 2-DG.

7. The pharmaceutical composition of claim 1, wherein the inhibitor of at least one of: PERK and GCN2 is a GCN2 inhibitor selected from the group consisting of: an siRNA directed against GCN2, glutamine, and an amino acid other than glutamine that inactivates or dephosphorylates GCN2.

8. The pharmaceutical composition of claim 1, wherein the cancer cells are growing under normoxia.

9. The pharmaceutical composition of claim 1, wherein the composition comprises an inhibitor of PERK and an inhibitor of GCN2.

10. The pharmaceutical composition of claim 9, wherein the PERK inhibitor is an siRNA directed against PERK and the GCN2 inhibitor is glutamine.

11. A method of treating cancer in a subject comprising: administering to the subject a therapeutically effective amount of a composition comprising a pharmaceutically acceptable carrier, a sugar analog in an amount effective for inhibiting the growth of cancer cells, and an inhibitor of at least one of: PERK and GCN2 in an amount effective for blocking phosphorylation of eif2-α in the cancer cells,
wherein the combined amounts of the sugar analog and the inhibitor of at least one of, PERK and GCN2 are sufficient for inducing death of the cancer cells.

12. The method of claim 11, wherein the subject is a mammal.

13. The method of claim 11, wherein the amount of the sugar analog effective for inhibiting the growth of cancer cells is sufficient for inducing ER stress in the cancer cells as measured by ER stress assays.

14. The method of claim 11, wherein the sugar analog is selected from the group consisting of: 2-DG, an analog of 2-DG, 2-FM, an analog of 2-FM, 2-BM, an analog of 2-BM, 2-CM, an analog of 2-CM, 2-DM, an analog of 2-DM, 2-FG, and an analog of 2-FG.

15. The method of claim 11, wherein the inhibitor of at least one of: PERK and GCN2 is a PERK inhibitor and the sugar analog is 2-DG.

16. The method of claim 15, wherein the PERK inhibitor is an siRNA directed against PERK.

17. The method of claim 11, wherein the inhibitor of at least one of: PERK and GCN2 is a GCN2 inhibitor and the sugar analog is 2-DG.

18. The method of claim 11, wherein the inhibitor of at least one of: PERK and GCN2 is a GCN2 inhibitor selected from the group consisting of: an siRNA directed against GCN2, glutamine, and an amino acid other than glutamine that inactivates or dephosphorylates GCN2.

19. The method of claim 11, wherein the cancer cells are growing under normoxia.

20. The method of claim 11, wherein the composition comprises an inhibitor of PERK and an inhibitor of GCN2.

21. The method of claim 20, wherein the PERK inhibitor is an siRNA directed against PERK and the GCN2 inhibitor is glutamine.

22. A kit for treating cancer in a subject, the kit comprising:
a) a composition comprising a pharmaceutically acceptable carrier, a sugar analog in an amount effective for inhibiting the growth of cancer cells, and an inhibitor of at least one of: PERK and GCN2 in an amount effective for blocking phosphorylation of eif2-α in the cancer cells, wherein the combined amounts of the sugar analog and the inhibitor of at least one of: PERK and GCN2 are sufficient for inducing death of the cancer cells;
b) packaging; and
c) instructions for use.

23. The kit of claim 22, wherein the amount of the sugar analog effective for inhibiting the growth of cancer cells is sufficient for inducing ER stress in the cancer cells as measured by ER stress assays.

24. The kit of claim 22, wherein the sugar analog is selected from the group consisting of: 2-DG, an analog of 2-DG, 2-FM, an analog of 2-FM, 2-BM, an analog of 2-BM, 2-CM, an analog of 2-CM, 2-DM, art analog of 2-DM, 2-FG, and an analog of 2-G.

25. The kit of claim 22, wherein the inhibitor of at least one of: PERK and GCN2 is a PERK inhibitor and the sugar analog is 2-DG.

26. The kit of claim 25, wherein the PERK inhibitor is an siRNA directed against PERK.

27. The kit of claim 22, wherein the inhibitor of at least one of: PERK and GCN2 is a GCN2 inhibitor and the sugar analog is 2-DG.

28. The kit of claim 22, wherein the inhibitor of at least one of: PERK and GCN2 is a GCN2 inhibitor selected from the group consisting of: glutamine, and an amino acid other than glutamine that inactivates or dephosphorylates GCN2.

29. The kit of claim 22, wherein the composition comprises an inhibitor of PERK and an inhibitor of GCN2.

30. The kit of claim 29, wherein the PERK inhibitor is an siRNA directed against PERK and the GCN2 inhibitor is glutamine.
